(19) United States
(12) Patent Application Publication
(10) Pub. No.: US 2026/0130615 A1
(43) Pub. Date: May 14, 2026

(54) METHOD AND APPARATUS FOR MONITORING SLEEP STATE AND ANALYZING MENTAL HEALTH

(71) Applicant: ELECTRONICS AND TELECOMMUNICATIONS RESEARCH INSTITUTE, Daejeon (KR)

(72) Inventors: Eun Kyoung Jeon, Daejeon (KR); Sehwan MOON, Daejeon (KR); Aram Lee, Daejeon (KR); Jeong Eun Kim, Daejeon (KR)

(21) Appl. No.: 19/385,543

(22) Filed: Nov. 11, 2025

(30) Foreign Application Priority Data
Nov. 12, 2024 (KR) 10-2024-0160152
Oct. 21, 2025 (KR) 10-2025-0152540

(51) Int. Cl. A61B 5/16; A61B 5/00

(52) U.S. Cl. CPC A61B 5/165; A61B 5/4809; A61B 5/7264

(57) ABSTRACT

Disclosed are a method and apparatus for monitoring a sleep state and analyzing mental health. The method includes determining whether a user is in a sleeplessness state based on activity data of the user, presenting, by a counselor module based on artificial intelligence (AI), a question for analyzing mental health of the user to the user when the user is in the sleeplessness state and receiving an answer responding to the question from the user, and generating a prompt for determining the severity of depression and anxiety states of the user by combining the question and the answer with a previously stored prompt template, obtaining the result of the analysis of mental health of the user by inputting the prompt to a large language model (LLM), and transmitting the result of the analysis of the mental health of the user to a terminal of the user.

### CROSS-REFERENCE TO RELATED APPLICATIONS

This application claims priority to and the benefit of Korean Patent Application No. 10-2024-0160152, filed on November 12, 2024, and Korean Patent Application No. 10-2025-0152540, filed on October 21, 2025, the disclosures of which are incorporated herein by reference in their entirety.

### BACKGROUND

### Technical Field

The present disclosure relates to a method and apparatus for monitoring a sleep state and analyzing mental health.

### Related Art

The existing sleep monitoring technique is basically focused on measuring the sleep pattern of a user through a smartphone or a wearable device. However, such techniques do not consider the influence of a change in the sleep state on mental health of a user or have limitations in accurately detecting an early sign of sleeplessness. In particular, although there is a good possibility that the sleeplessness state may lead to mental health problems, such as depression and uneasiness, when the sleeplessness state continues, a system capable of diagnosing and intervening the sleeplessness state at an early stage is not sufficient.

### SUMMARY

Various embodiments are directed to proposing an apparatus and method for monitoring a sleep state in real time based on the activity data of a smartphone, unconsciousnessly detecting a sleeplessness state at an early stage, and providing quick alert alarm if necessary.

Furthermore, various embodiments are directed to proposing an apparatus and method for analyzing the depression and anxiety states of a user through an artificial intelligence (AI) counselor and a large language model (LLM) when the sleeplessness state of the user continues and checking the severity of the depression and anxiety states so that mental health can be managed more comprehensively.

Objects of the present disclosure are not limited to the aforementioned object, and other objects not described above may be evidently understood by those skilled in the art from the following description.

A method of monitoring a sleep state and analyzing mental health according to an embodiment of the present disclosure may be performed by an apparatus for monitoring a sleep state and analyzing mental health according to an embodiment of the present disclosure.

The method of monitoring a sleep state and analyzing mental health includes determining whether a user is in a sleeplessness state based on activity data of the user, presenting, by a counselor module based on artificial intelligence (AI), a question for analyzing mental health of the user to the user when the user is in the sleeplessness state and receiving an answer responding to the question from the user, and generating a prompt for determining the severity of depression and anxiety states of the user by combining the question and the answer with a previously stored prompt template, obtaining the result of the analysis of mental health of the user by inputting the prompt to a large language model (LLM), and transmitting the result of the analysis of the mental health of the user to a terminal of the user.

The method of monitoring a sleep state and analyzing mental health may further include collecting the activity data from the terminal.

The activity data may include step data of the user measured by a sensor mounted on the terminal.

The step data may include average daily steps of the user and split-timed average steps of the user.

In an embodiment of the present disclosure, the determining of whether the user is in the sleeplessness state may include determining whether the user is in the sleeplessness state based on a change pattern of the activity data for a predetermined period.

In an embodiment of the present disclosure, the determining of whether the user is in the sleeplessness state may include determining whether the user is in the sleeplessness state by inputting the activity data for a predetermined period to a pre-trained sleep state analysis model based on artificial intelligence (AI).

The method of monitoring a sleep state and analyzing mental health may further include transmitting warning to the terminal when the severity of the depression and anxiety states of the user is greater than a predetermined threshold based on the result of the analysis of the mental health.

An apparatus for monitoring a sleep state and analyzing mental health according to an embodiment of the present disclosure includes a processor and memory in which one or more instructions executed by the processor are stored.

The one or more instructions include an instruction to determine whether a user is in a sleeplessness state based on activity data of the user, an instruction to present, by a counselor module based on artificial intelligence (AI), a question for analyzing mental health of the user to the user when the user is in the sleeplessness state and receive an answer responding to the question from the user, and an instruction to generate a prompt for determining the severity of depression and anxiety states of the user by combining the question and the answer with a previously stored prompt template, obtain the result of the analysis of mental health of the user by inputting the prompt to a large language model (LLM), and transmit the result of the analysis of the mental health of the user to a terminal of the user.

The one or more instructions may further include an instruction to collect the activity data from the terminal.

The activity data may include step data of the user measured by a sensor mounted on the terminal.

In an embodiment of the present disclosure, the step data may include average daily steps of the user and split-timed average steps of the user.

In an embodiment of the present disclosure, the instruction to determine whether the user is in the sleeplessness state may include an instruction to determine whether the user is in the sleeplessness state based on a change pattern of the activity data for a predetermined period.

In an embodiment of the present disclosure, the instruction to determine whether the user is in the sleeplessness state may include an instruction to determine whether the user is in the sleeplessness state by inputting the activity data for a predetermined period to a pre-trained sleep state analysis model based on artificial intelligence (AI).

In an embodiment of the present disclosure, the one or more instructions may further include an instruction to transmit warning to the terminal when the severity of the depression and anxiety states of the user is greater than a predetermined threshold based on the result of the analysis of the mental health.

According to embodiments of the present disclosure, it is possible to analyze mental health precisely compared to the existing simple sleep monitoring system because the sleeplessness state can be detected at an early stage based on the activity data of a smartphone and the depression and anxiety states can be comprehensively evaluated by using an AI counselor and a large AI model.

According to embodiments of the present disclosure, the early warning of the sleeplessness state and customized mental health management are made possible. Accordingly, it is possible to contribute to comprehensive well-being improvements by providing a user with preventive management and appropriate intervention for a mental health problem.

Effects of the present disclosure which may be obtained in the present disclosure are not limited to the aforementioned effects, and other effects not described above may be evidently understood by a person having ordinary knowledge in the art to which the present disclosure pertains from the following description.

### BRIEF DESCRIPTION OF THE DRAWINGS

FIG. 1 is a block diagram illustrating a physical construction of an apparatus for monitoring a sleep state and analyzing mental health according to an embodiment of the present disclosure.

FIG. 2 is a block diagram illustrating a functional construction of the apparatus for monitoring a sleep state and analyzing mental health according to an embodiment of the present disclosure.

FIG. 3 is a diagram illustrating an example of an operation of the apparatus for monitoring a sleep state and analyzing mental health according to an embodiment of the present disclosure.

FIG. 4 is a flowchart for describing a method of monitoring a sleep state and analyzing mental health according to an embodiment of the present disclosure.

### DETAILED DESCRIPTION

Embodiments of the present disclosure relate to a method and apparatus for monitoring a sleep state and analyzing mental health based on the activity data of a smartphone.

Embodiments of the present disclosure relate to a method and apparatus for monitoring a change in the sleep state based on the activity data of a user smartphone, detecting the sleeplessness state at an early stage, and analyzing depression and anxiety states based on counseling contents through activity analysis and an artificial intelligence (AI) counselor when the sleeplessness state occurs. In particular, embodiments of the present disclosure relate to a method and apparatus for unconsciousnessly monitoring the active state and sleep state of a user based on the activity data of a smartphone, classifying the depression and anxiety states through simple consultations, and checking the severity of the depression and anxiety states.

Advantages and characteristics of the present disclosure and a method for achieving the advantages and characteristics will become apparent from embodiments described in detail later in conjunction with the accompanying drawings. However, the present disclosure is not limited to the disclosed embodiments, but may be implemented in various different forms. The embodiments are merely provided to complete the present disclosure and to fully notify a person having ordinary knowledge in the art to which the present disclosure pertains to the category of the present disclosure. The present disclosure is merely defined by the category of the claims. Terms used in this specification are used to describe embodiments and are not intended to limit the present disclosure. In this specification, an expression of the singular number includes an expression of the plural number unless clearly defined otherwise in the context. The term “comprises” and/or “comprising” used in this specification does not exclude the presence or addition of one or more other components, steps, operations and/or components in addition to mentioned components, steps, operations and/or components.

Terms, such as a first and a second, may be used to describe various components, but the components should not be restricted by the terms. The terms may be used to only distinguish one component from the other components. Accordingly, a first component may be named a second component without departing from the scope of a right of the present disclosure. Likewise, a second component may also be named a first component.

When it is described that one component is “connected” or “coupled” to the other component, it should be understood that one component may be directly connected or coupled to the other component, but a third component may exist between the two components. In contrast, when it is described that one component is “directly connected to” or “directly coupled to” the other component, it should be understood that a third component does not exist between the two components. Other expressions for describing relations between components, that is, “between ~”, “just between ~”, “adjacent to ~”, and “neighboring ~”, should be likewise construed.

In describing the present disclosure, a detailed description of a related known technology will be omitted if it is deemed to make the subject matter of the present disclosure unnecessarily vague.

Hereinafter, embodiments of the present disclosure are described in detail with reference to the accompanying drawings. In describing the present disclosure, in order to facilitate general understanding of the present disclosure, the same reference numeral is used for the same mean regardless of the reference numeral.

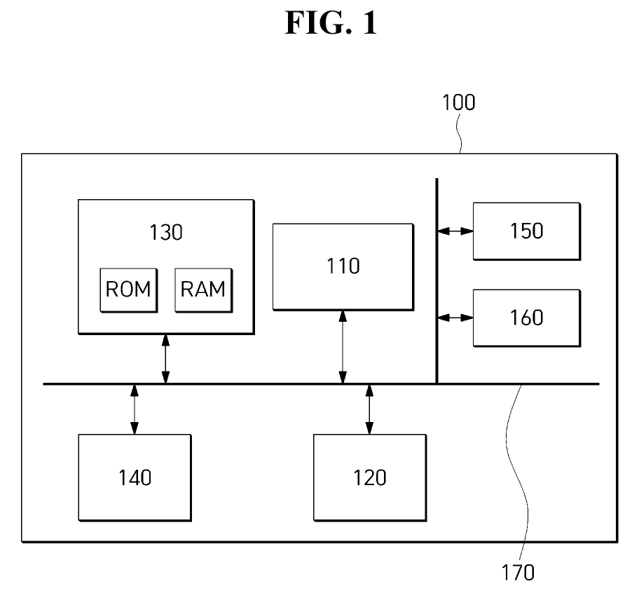

FIG. 1 is a block diagram illustrating a physical construction of an apparatus for monitoring a sleep state and analyzing mental health according to an embodiment of the present disclosure.

As illustrated in FIG. 1, an apparatus 100 for monitoring a sleep state and analyzing mental health according to an embodiment of the present disclosure may be implemented in the form of a computer system.

For reference, unlike in FIG. 1, the apparatus 100 for monitoring a sleep state and analyzing mental health according to an embodiment of the present disclosure may be implemented in the form of software, or hardware, such as a field programmable gate array (FPGA) or an application-specific integrated circuit (ASIC).

Referring to FIG. 1, the apparatus 100 for monitoring a sleep state and analyzing mental health according to an embodiment of the present disclosure includes a processor 110, a communication device 120, memory 130, a storage device 140, an input interface device 150, an output interface device 160, and a bus 170. The apparatus 100 for monitoring a sleep state and analyzing mental health, which is illustrated in FIG. 1, corresponds to an embodiment. Components of the apparatus 100 for monitoring a sleep state and analyzing mental health according to an embodiment of the present disclosure are not limited to the embodiment illustrated in FIG. 1, and a component may be added to the components or a component may be changed or deleted, if necessary.

The processor 110 may be a central processing unit (CPU) or may be a semiconductor device that executes a computer-readable instruction stored in the memory 130 or the storage device 140. The memory 130 and the storage device 140 may each include various types of volatile or non-volatile storage media. For example, the memory 130 may include read only memory (ROM) and random access memory (RAM). In an embodiment of the present specification, the memory 130 may be disposed inside or outside the processor 110 and connected to the processor 110 through various known means. The memory 130 includes various types of volatile or nonvolatile storage media, and may include ROM or RAM, for example.

Accordingly, an embodiment of the present disclosure may be implemented as a method implemented in a computer or may be implemented as a non-transitory computer-readable medium in which a computer-executable instruction has been stored. In an embodiment, when being executed by the processor 110, a computer-readable instruction may perform a method according to at least one aspect of this writing.

The communication device 120 may transmit or receive a wired signal or a wireless signal.

Furthermore, an operating method of the apparatus 100 for monitoring a sleep state and analyzing mental health according to an embodiment of the present disclosure may be implemented in the form of a program instruction which may be executed through various computer means, and may be recorded on a computer-readable medium.

The computer-readable medium may include a program instruction, a data file, and a data structure alone or in combination. A program instruction recorded on the computer-readable medium may be specially designed and constructed for an embodiment of the present disclosure or may be known and available to those skilled in the computer software field. The computer-readable medium may include a hardware device configured to store and execute the program instruction. For example, the computer-readable medium may include magnetic media such as a hard disk, a floppy disk, and a magnetic tape, optical media such as CD-ROM and a DVD, magneto-optical media such as a floptical disk, ROM, RAM, and flash memory. The program instruction may include not only a machine code produced by a compiler, but a high-level language code capable of being executed by a computer through an interpreter.

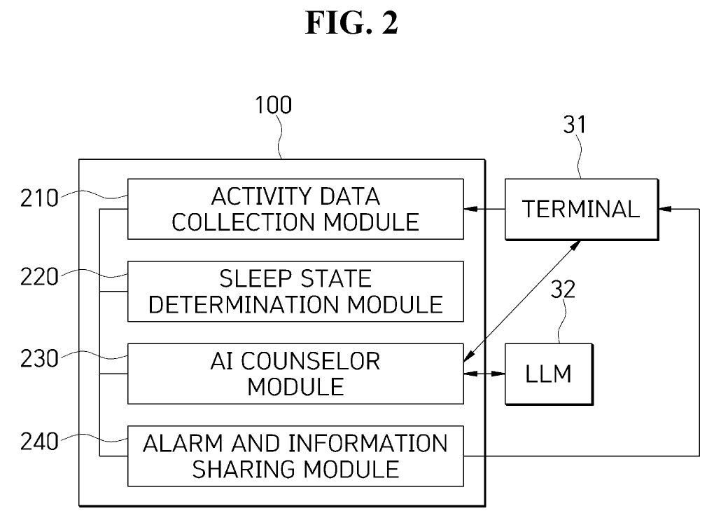

The processor 110 executes the method of monitoring a sleep state and analyzing mental health by executing computer-readable instructions that are stored in the memory 130 or the storage device 140. Detailed contents of embodiments of the present disclosure are described later with reference to FIGS. 2 to 4.

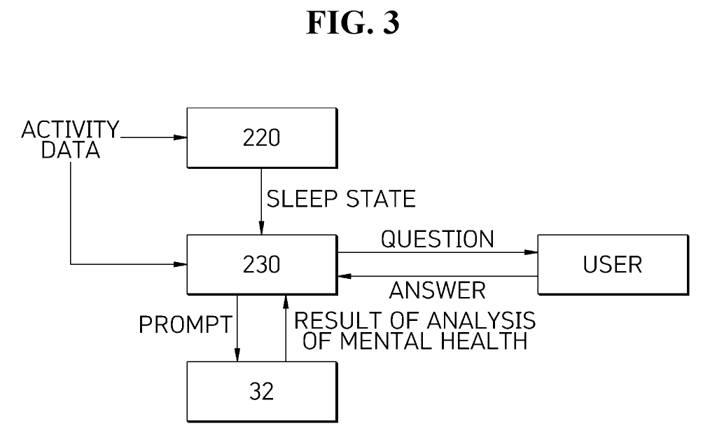

FIG. 2 is a block diagram illustrating a functional construction of the apparatus for monitoring a sleep state and analyzing mental health according to an embodiment of the present disclosure. FIG. 3 is a diagram illustrating an example of an operation of the apparatus for monitoring a sleep state and analyzing mental health according to an embodiment of the present disclosure.

As illustrated in FIG. 2, the apparatus 100 for monitoring a sleep state and analyzing mental health may include an activity data collection module 210, a sleep state determination module 220, an artificial intelligence (AI) counselor module 230, and an alarm and information sharing module 240.

The activity data collection module 210 collects the activity data of a user. For example, the activity data collection module 210 receives the activity data from a terminal 31 of a user. For example, the terminal 31 may be a mobile device, such as a smartphone that is carried by a user.

For example, the activity data may include the step data of a user that are measured by a sensor mounted on the terminal 31. The step data may include average daily steps of the user and split-timed average steps of the user.

As another example, the activity data may include frequency and intensity (e.g., may be measured by an accelerometer), a location, trajectory, and a distance of a movement, voice (e.g., including the sound of breathing or snoring during sleep) of a user, and illuminance (may be measured by an illuminance sensor).

The sleep state determination module 220 determines whether the user is in the sleeplessness state based on the activity data collected by the activity data collection module 210. For example, the sleep state determination module 220 may determine whether the user is in the sleeplessness state based on a change pattern of the activity data for a predetermined period (e.g., 1 day).

As another example, the sleep state determination module 220 may determine whether the user is in the sleeplessness state by inputting the activity data for a predetermined period to a pre-trained sleep state analysis model based on AI.

When determining that the user is in the sleeplessness state, the sleep state determination module 220 operates the AI counselor module 230.

The AI counselor module 230 performs additional diagnosis because the user is in the sleeplessness state. The AI counselor module 230 generates a question (hereinafter referred to as a “consultation question”) for analyzing mental health of the user based on AI and presents the question to the user. Furthermore, the AI counselor module 230 receives an answer responding to the consultation question from the user. For example, the AI counselor module 230 may generate the consultation question by inputting the activity data of the user to an artificial neural network model that has been trained by using the activity data and psychiatric consultation data as training data.

In this case, the consultation question may include the activity data, the result of the analysis of the activity data, or the result of a determination of the sleep state (e.g., the sleeplessness state) of the user. For example, the consultation question may be generated in a form, such as “Unusually, you’ve taken more than 200 steps after 12 o’clock, and you seem to have trouble sleeping. I’d like to know how you feel.”

For example, the AI counselor module 230 may transmit a consultation question that is generated in text to the terminal 31 of the user, and may receive the answer of the user from the terminal 31.

As another example, the AI counselor module 230 may pronounce a consultation question that is generated in a voice and receive an answer voice pronounced by the user. In this case, the AI counselor module 230 converts the answer voice into text by using a preset voice recognition model. Furthermore, the AI counselor module 230 may extract the voice features of the user by inputting the answer voice to a preset voice feature extraction model.

Furthermore, the AI counselor module 230 generates a prompt that determines the severity of depression and anxiety states of the user by combining the consultation question and the answer of the user corresponding to the consultation question with a previously stored prompt template. The AI counselor module 230 obtains the result of the analysis of mental health of the user by inputting the generated prompt to a large language model (LLM) 32, and transmits the result of the analysis of the mental health including the severity of the depression and anxiety states to the terminal 31 of the user or a terminal (not illustrated) of an interested party (e.g., a family doctor or a family) of the user through the alarm and information sharing module 240.

As described above, the result of the analysis of the mental health includes the severity (e.g., scales from 0 to 24) of the depression and anxiety states. The apparatus 100 for monitoring a sleep state and analyzing mental health may include the LLM 32 or may use the LLM 32 within an external server.

The prompt may include additional information in addition to the consultation question, the answer, and the preset template in order to assist the LLM in making a determination. For example, the AI counselor module 230 may include the voice features extracted from the answer voice of the user in the prompt. For example, the voice features may include a frequency, a ratio of a pause to an utterance time, a pitch, a noise ratio, the irregularity of vocal cord vibration, Mel frequency cepstral coefficients (MFCC), intensity, and a volume change rate.

Furthermore, the template included in the prompt may include an instruction to instruct the LLM to include severity according to a predetermined criterion in the result of the analysis of the mental health. For example, the criterion may be personal health questionnaire (PHQ) depression scale-8.

When the severity (e.g., 20 on the basis of 24 scores) included in the result of the analysis of the mental health is greater than a threshold (e.g., 19), the AI counselor module 230 transmits a warning message, including the result of the analysis of the mental health and the severity, to the terminal 31 of the user, a terminal of an interested party (e.g., a family doctor or a family) of the user, or the user through the alarm and information sharing module 240.

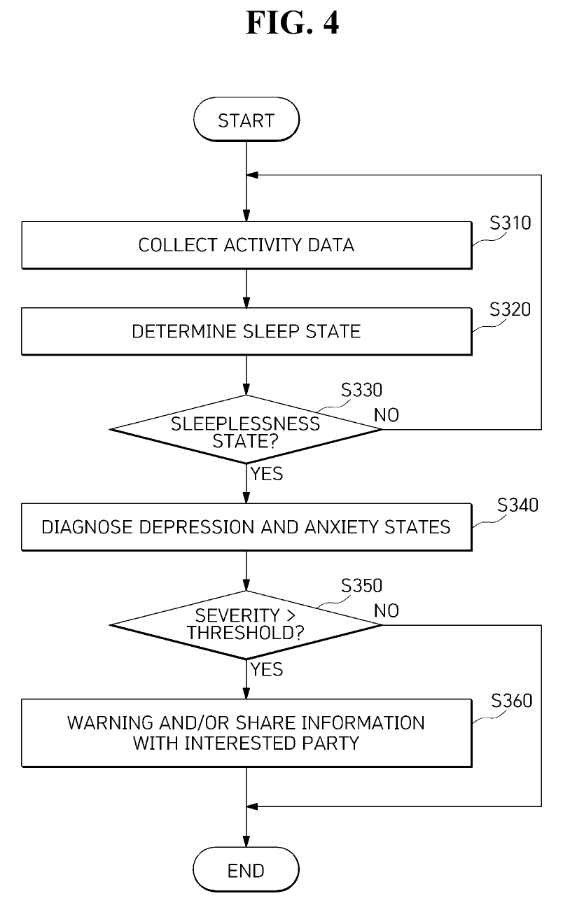

FIG. 4 is a flowchart for describing a method of monitoring a sleep state and analyzing mental health according to an embodiment of the present disclosure.

Referring to FIG. 4, the method of monitoring a sleep state and analyzing mental health according to an embodiment of the present disclosure includes steps S310 to S360. The method of monitoring a sleep state and analyzing mental health, which is illustrated in FIG. 4, corresponds to an embodiment. Steps of the method of monitoring a sleep state and analyzing mental health according to an embodiment of the present disclosure are not limited to the embodiment illustrated in FIG. 4, and a step may be added to the steps or a step may be changed or deleted, if necessary.

For convenience’ sake, it is presupposed that the method of monitoring a sleep state and analyzing mental health, which is illustrated in FIG. 4, is performed by the apparatus 100 for monitoring a sleep state and analyzing mental health.

Step S310 is an activity data collection step.

The processor 110 of the apparatus 100 for monitoring a sleep state and analyzing mental health collects the activity data of a user through the communication device 120 or the input interface device 150. In an embodiment of the present disclosure, the activity data refer to data that provide a basis on which an active pattern or sleep state of a user may be determined.

For example, the communication device 120 of the apparatus 100 for monitoring a sleep state and analyzing mental health receives the activity data from the terminal 31 of the user and transmits the collected activity data to the processor 110.

For example, the activity data include the step data and movement frequency of the user measured by a sensor mounted on the terminal 31. The step data may include average daily steps of the user and split-timed average steps of the user.

Step S320 is a sleep state determination step.

The processor 110 determines whether the user is in the sleeplessness state based on the activity data.

For example, the processor 110 may determine whether the user is in the sleeplessness state based on a change pattern of the activity data for a predetermined period (e.g., 1 day).

As another example, the processor 110 may determine whether the user is in the sleeplessness state by inputting the activity data for a predetermined period to a pre-trained sleep state analysis model based on AI.

Step S330 is a step that is divided depending on the sleeplessness state.

When determining that the user is in the sleeplessness state in step S320, the apparatus 100 for monitoring a sleep state and analyzing mental health proceeds to step S340. If not, the apparatus 100 for monitoring a sleep state and analyzing mental health returns to step S310.

Step S340 is depression and anxiety states diagnosis step.

The apparatus 100 for monitoring a sleep state and analyzing mental health performs additional diagnosis because the user is in the sleeplessness state. The processor 110 generates a question (hereinafter referred to as a “consultation question”) for analyzing mental health of the user based on AI and presents the question to the user through the communication device 120 or the output interface device 160. Furthermore, the processor 110 receives an answer responding to the consultation question from the user through the communication device 120 or the input interface device 150. For example, the processor 110 may generate the consultation question by inputting the activity data of the user to an artificial neural network model that has been trained by using the activity data and psychiatric consultation data as training data.

In this case, the consultation question may include the activity data, the result of the analysis of the activity data, or the result of the determination of a sleep state (e.g., the sleeplessness state) of the user. For example, the consultation question may be generated in a form, such as “Unusually, you’ve taken more than 200 steps after 12 o’clock, and you seem to have trouble sleeping. I’d like to know how you feel.”

For example, the processor 110 may transmit a consultation question that is generated in text to the terminal 31 of the user through the communication device 120, and may receive the answer of the user from the terminal 31.

As another example, the processor 110 may pronounce a consultation question that is generated in voice through the output interface device 160, and may receive an answer voice pronounced by the user through the input interface device 150. In this case, the output interface device 160 may be a speaker, and the input interface device 150 may be a microphone. In this case, the processor 110 converts the answer voice into text by using a preset voice recognition model. The processor 110 may extract the voice features of the user by inputting the answer voice to a preset voice feature extraction model.

In this step, the processor 110 generates a prompt that determines the severity of depression and anxiety states of the user by combining the consultation question and the answer of the user corresponding to the consultation question with a previously stored prompt template. The processor 110 obtains the result of the analysis of mental health of the user by inputting the generated prompt to the LLM 32, and transmits the result of the analysis of mental health of the user to the terminal 31 of the user.

The result of the analysis of the mental health includes the severity (e.g., scales from 0 to 24) of the depression and anxiety states. The apparatus 100 for monitoring a sleep state and analyzing mental health may include the LLM 32 or may use the LLM 32 within an external server.

The prompt may include additional information in addition to the consultation question, the answer, and the preset template in order to help the LLM make a determination. For example, the processor 110 may include the voice features extracted from the answer voice of the user in the prompt. For example, the voice features may include a frequency, a ratio of a pause to an utterance time, a pitch, a noise ratio, the irregularity of vocal cord vibration, Mel frequency cepstral coefficients (MFCC), intensity, and a volume change rate.

Furthermore, the template included in the prompt may include an instruction to instruct the LLM to include severity according to a predetermined criterion in the result of the analysis of the mental health. For example, the criterion may be personal health questionnaire (PHQ) depression scale-8.

Step S350 is a step of determining whether the severity of the depression and anxiety states of the user is greater than a predetermined threshold. Step S360 is a warning step.

When the severity (e.g., 20 on the basis of 24 scores) included in the result of the analysis of the mental health is greater than the threshold (e.g., 19), the processor 110 transmits a warning message, including the result of the analysis of the mental health and the severity, to the terminal 31 of the user, a terminal of an interested party (e.g., a family doctor or a family) of the user, or the user through the communication device 120 or the output interface device 160.

The method of monitoring a sleep state and analyzing mental health has been described with reference to the flowcharts presented in the drawings. For a simple description, the method has been illustrated and described as a series of blocks, but the present disclosure is not limited to the sequence of the blocks, and some blocks may be performed in a sequence different from or simultaneously with that of other blocks, which has been illustrated and described in this specification. Various other branches, flow paths, and sequences of blocks which achieve the same or similar results may be implemented. Furthermore, all the blocks illustrated in order to implement the method described in this specification may not be required.

In the description given with reference to FIG. 4, each of the steps may be further divided into additional steps or the steps may be combined into smaller steps depending on an implementation example of the present disclosure. Furthermore, some of the steps may be omitted, if necessary, and the sequence of the steps may be changed. Furthermore, the contents of FIGS. 1 to 3, although some contents are omitted, may be applied to the contents of FIG. 4. Furthermore, the contents of FIG. 4 may be applied to the contents of FIGS. 1 to 3.

Although the present disclosure has been described with reference to the preferred embodiments, those skilled in the art may understand that the present disclosure may be modified and changed in various ways without departing from the spirit and scope of the present disclosure written in the claims.

### Description of reference numerals

31: terminal 32: LLM

100: apparatus for monitoring sleep state and analyzing mental health

110: processor 120: communication device

130: memory 140: storage device

150: input interface device

160: output interface device

170: bus 210: activity data collection module

220: sleep state determination module

230: AI counselor module

240: alarm and information sharing module

What is claimed is:

1. A method of monitoring a sleep state and analyzing mental health, the method being performed by an apparatus for monitoring a sleep state and analyzing mental health and comprising:
determining whether a user is in a sleeplessness state based on activity data of the user;
presenting a question for analyzing mental health of the user to the user when the user is in the sleeplessness state and receiving an answer responding to the question from the user; and
generating a prompt for determining a severity of depression and anxiety states of the user by combining the question and the answer with a previously stored prompt template, obtaining a result of an analysis of mental health of the user by inputting the prompt to a large language model (LLM), and transmitting the result of the analysis of the mental health of the user to a terminal of the user.

2. The method of claim 1, further comprising collecting the activity data from the terminal, wherein the activity data comprise step data of the user measured by a sensor mounted on the terminal.

3. The method of claim 2, wherein the step data comprise average daily steps of the user and split-timed average steps of the user.

4. The method of claim 1, wherein the determining of whether the user is in the sleeplessness state comprises determining whether the user is in the sleeplessness state based on a change pattern of the activity data for a predetermined period.

5. The method of claim 1, wherein the determining of whether the user is in the sleeplessness state comprises determining whether the user is in the sleeplessness state by inputting the activity data for a predetermined period to a pre-trained sleep state analysis model based on artificial intelligence (AI).

6. The method of claim 1, further comprising transmitting warning to the terminal when the severity of the depression and anxiety states of the user is greater than a predetermined threshold based on the result of the analysis of the mental health.

7. An apparatus for monitoring a sleep state and analyzing mental health, the apparatus comprising:
a processor; and
memory in which one or more instructions executed by the processor are stored, and
wherein the one or more instructions comprise:
an instruction to determine whether a user is in a sleeplessness state based on activity data of the user;
an instruction to present a question for analyzing mental health of the user to the user when the user is in the sleeplessness state and receive an answer responding to the question from the user; and
an instruction to generate a prompt for determining a severity of depression and anxiety states of the user by combining the question and the answer with a previously stored prompt template, obtain a result of an analysis of mental health of the user by inputting the prompt to a large language model (LLM), and transmit the result of the analysis of the mental health of the user to a terminal of the user.

8. The apparatus of claim 7, wherein the one or more instructions further comprise an instruction to collect the activity data from the terminal, wherein the activity data comprise step data of the user measured by a sensor mounted on the terminal.

9. The apparatus of claim 8, wherein the step data comprise average daily steps of the user and split-timed average steps of the user.

10. The apparatus of claim 7, wherein the instruction to determine whether the user is in the sleeplessness state comprises an instruction to determine whether the user is in the sleeplessness state based on a change pattern of the activity data for a predetermined period.

11. The apparatus of claim 7, wherein the instruction to determine whether the user is in the sleeplessness state comprises an instruction to determine whether the user is in the sleeplessness state by inputting the activity data for a predetermined period to a pre-trained sleep state analysis model based on artificial intelligence (AI).

12. The apparatus of claim 7, wherein the one or more instructions further comprise an instruction to transmit warning to the terminal when the severity of the depression and anxiety states of the user is greater than a predetermined threshold based on the result of the analysis of the mental health.
